(19) United States
(12) Patent Application Publication
(10) Pub. No.: US 2019/0217026 A1
(43) Pub. Date: Jul. 18, 2019

(54) SPRAYING EQUIPMENT WITH DOUBLE-WATER-TANK STRUCTURE

(71) Applicant: Shenzhen Shenan Yangguang Electronics Co., Ltd, Shenzhen (CN)

(72) Inventor: Binglei Ye, Shenzhen (CN)

(21) Appl. No.: 15/871,152

(22) Filed: Jan. 15, 2018

(51) Int. Cl. A61M 11/00; B05B 17/06; B05B 7/00; A61L 9/14

(52) U.S. Cl. CPC A61M 11/005; B05B 17/06; A61L 2209/132; A61L 9/14; A61L 2209/11; B05B 7/0081

(57) ABSTRACT

Spraying equipment with a double-water-tank structure has two sets of independent spraying equipment, a wireless device and a control device are connected to the spraying equipment at the same time, the equipment can be controlled by upper and lower sub-equipment control software (e.g., equipment APP) of an intelligent terminal, meanwhile after other intelligent equipment forms a wireless connection with the equipment, the collected data of the other intelligent equipment are analyzed to control the operation of the equipment. For example, the data connection of the Amazon ECHO and the Google HOME and other intelligent sound boxes is supported to analyze the voice of a user and control the operation of the equipment. Meanwhile, the equipment is further provided with a manual control function and can select two or more control functions so as to satisfy the use effects under different environments.

### FIELD OF THE INVENTION

The present invention relates to the field of electronic equipment manufacturing, and in particular to the manufacturing of spraying equipment with a double-water-tank structure.

### BACKGROUND OF THE INVENTION

At present, a large amount of spraying equipment exist in the market, for example, an aromatherapy machine is a kind of spraying equipment which decomposes water molecules and dissolved plant essential oils into nanoscale cold fog with a diameter of 0.1-5 microns via high frequency oscillation (the oscillation frequency is 1.7 MHz or 2.4 MHz, which exceeds the hearing range of human beings, and thus the electronic oscillation generates no harm to the human body and animals at all) generated by ultrasonic wave oscillation equipment, and disperses the cold fog in the surrounding air, so that the air is filled with fragrance.

At present, the traditional aromatherapy machine on the market has only one set of ultrasonic atomization device, if the type of essential oil needs to be changed, the equipment should be thoroughly cleaned before use, and at the same time, different types of essential oils may produce unpleasant taste after being mixed and used. Therefore, for the users, the operation steps of the traditional spraying equipment are complicated, the function is relatively single, and the operability is poor. Furthermore, because of the only one set of ultrasonic atomization device, the atomization volume is generally smaller, and the humidification effect is limited. Meanwhile, the circuit regulation function of the traditional spraying equipment is single, the products of other companies on the market cannot be adjusted by a single mode, the atomization experience and the service life may be further improved, and there is a certain difference with the product of the present company.

### SUMMARY OF THE INVENTION

The objective of the present invention is to provide spraying equipment with a double-water-tank structure, which is simple in structure, wide in application and is mutually connected with an intelligent terminal.

To achieve the above invention purpose, the present invention adopts the following technical solution:

Spraying equipment with a double-water-tank structure includes an upper shell and a lower shell, wherein two air outlets are formed in the top of the upper shell; two air inlets are formed in the bottom of the lower shell, a redundant space is arranged between the upper shell and the lower shell, the double-water-tank structure, a double-atomization-sheet module and double fans are arranged in the redundant space, meanwhile the two air outlets in the upper shell and the two air inlets in the lower shell form two sets of independent aromatherapy spraying equipment together; a control motherboard, a control device, a wireless device, a light guide plate and control keys are further arranged in the redundant space; the double-water-tank structure is arranged at an upper part of the control motherboard, the double-atomization-sheet module, the control device and the wireless device are installed on the control motherboard, and the double fans are installed at a lower part of the double-atomization-sheet module; and the control device is connected with the double-atomization-sheet module, the double fans, the wireless device, the light guide plate and the control keys, controls the equipment and achieves a variety of control functions.

Preferably, the wireless device is connected with an intelligent control terminal, controls the equipment through system control software or other equipment software, and achieves a variety of control functions.

Preferably, the control keys are composed of two or more keys, the control keys are touch keys, and different touch keys are touched to control the equipment and achieve a variety of control functions.

Preferably, the double-atomization-sheet module generates ultrasonic waves after being energized, and the ultrasonic waves convert liquid substance in the water tanks into fog.

Preferably, the fans are located below the water tanks, thereby being conducive to guiding the flow of the fog in the water tanks along the direction of the air outlets.

Preferably, the light guide plate is connected with the control device and achieves different lamplight effects under different control functions so as to create environments with different atmospheres.

Preferably, the equipment further includes a power supply input port formed in the lower shell for charging the power supply of the equipment or directly providing power supply for the equipment, foot pads are arranged at the bottom of the equipment so as to beneficially improve the air inlet effect of the air inlets, meanwhile antiskid structures are arranged outside the foot pads to improve the product stability.

### BRIEF DESCRIPTION OF THE DRAWINGS

FIG. 1 is a first schematic diagram of the present art;

FIG. 2 is a second schematic diagram of the present art;

FIG. 3 is a third schematic diagram of the present art;

FIG. 4 is a first flowchart of the present art;

FIG. 5 is a second flowchart of the present art.

### DETAILED DESCRIPTION OF THE EMBODIMENTS

In order that the purposes, technical solutions and advantages of the present invention are clearer, a further detailed description of the present invention will be given below in combination with drawings and embodiments. It should be understood that the specific embodiments described herein are merely used for explaining the present invention, rather than limiting the present invention.

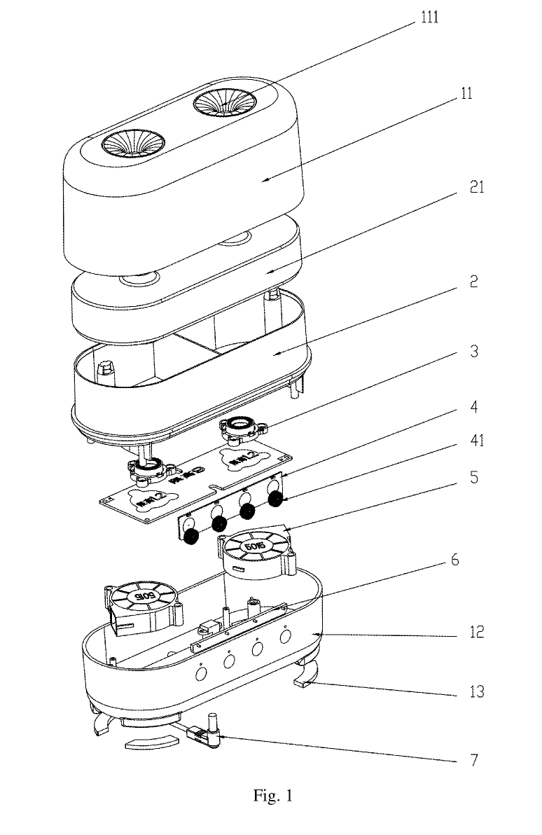

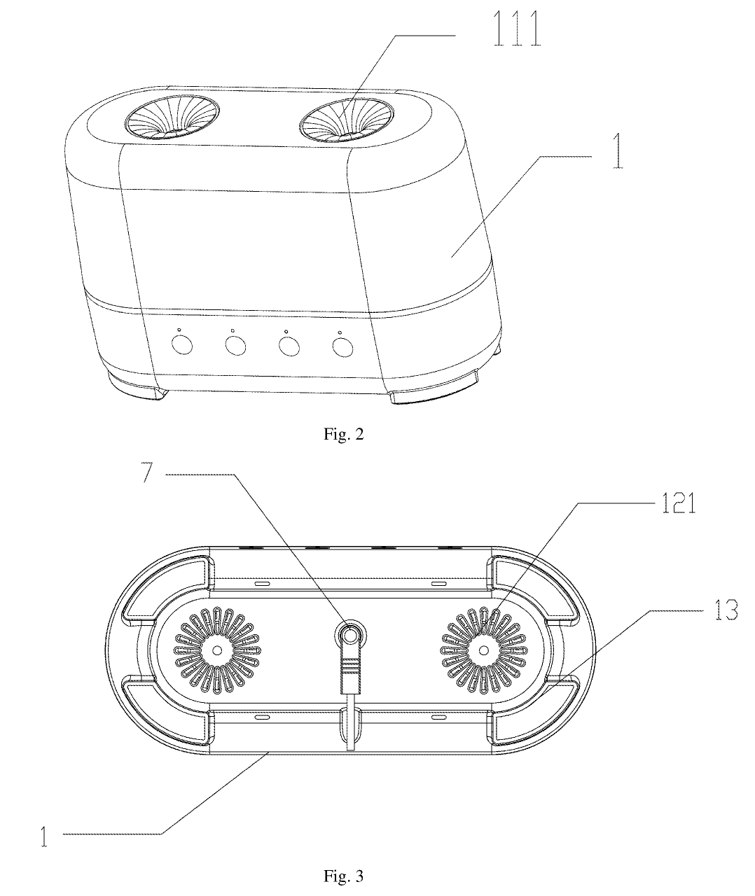

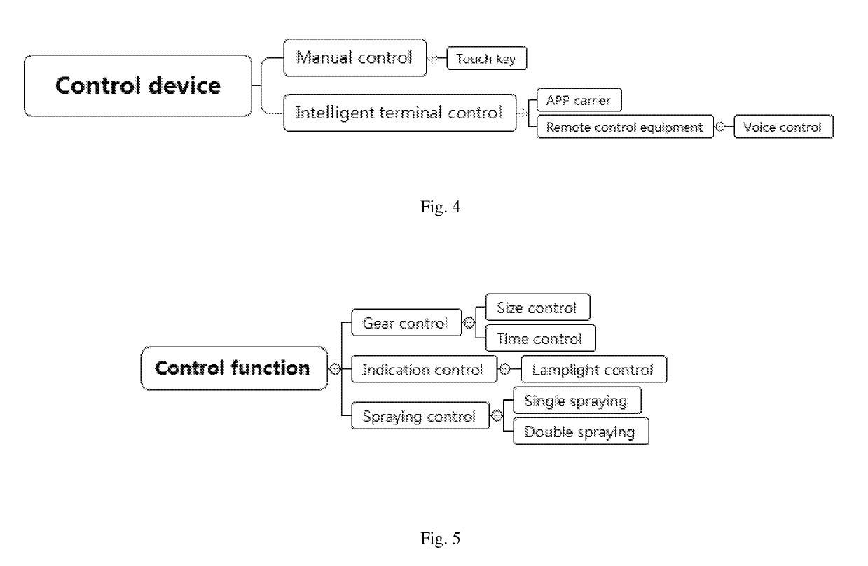

Referring to FIGS. 1-5, spraying equipment with a double-water-tank structure includes an upper shell 11 and a lower shell 12; wherein two air outlets 111 are formed in the top of the upper shell 11; two air inlets 121 are formed in the bottom of the lower shell 12, a redundant space is arranged between the upper shell 11 and the lower shell 12, the double-water-tank structure 2, a double-atomization-sheet module 3 and double fans 5 are arranged in the redundant space, meanwhile the two air outlets 111 in the upper shell and the two air inlets 121 in the lower shell form two sets of independent aromatherapy spraying equipment together; a control motherboard 4, a control device 7, a wireless device, a light guide plate 6 and control keys 41 are further arranged in the redundant space; the double-water-tank structure 2 is arranged at an upper part of the control motherboard, the double-atomization-sheet module 3, the control device and the wireless device are installed on the control motherboard, and the double fans are installed at a lower part of the double-atomization-sheet module; and the control device 4 is connected with the double-atomization-sheet module 3, the double fans 5, the wireless device, the light guide plate 6 and the control keys 41, controls the equipment and achieves a variety of control functions.

Preferably, the wireless device is connected with an intelligent control terminal, controls the equipment through system control software or other equipment software, and achieves a variety of control functions, for example, the intelligent control terminal such as a mobile phone and a tablet personal computer downloads an APP and connects the equipment via the wireless device and controls the equipment, as another example, in cooperation with the Amazon ECHO and the Google HOME and other intelligent sound box equipment, the equipment is connected via the wireless device, and the equipment is controlled by voice.

Preferably, the control keys 41 are composed of two or more keys, the control keys 41 are touch keys, and different touch keys are touched to control the equipment and achieve a variety of control functions. The control functions of the equipment mainly include gear control, indication control and spraying control, wherein the gear control includes the gear control at multiple time, the control of spraying size and other functions; and the spraying control can achieve single spraying and double spraying.

Preferably, the double-atomization-sheet module 3 generates ultrasonic waves after being energized, and the ultrasonic waves convert liquid substance in the water tanks into fog.

Preferably, the fans are located below the water tanks, thereby being conducive to guiding the flow of the fog in the water tanks along the direction of the air outlets. The size of the sprayed air blown by the fans directly determines the spraying size, generally, the air inlets 121 are located below the fan ports, the air outlets 111 are located above the fan ports, and the air outlets 111 can also be formed in the side face of the shell.

Preferably, the light guide plate 6 is connected with the control device and achieves different lamplight effects under different control functions so as to create environments with different atmospheres. The light guide plate is matched with a light source in the equipment and projects the light source to the outside of the equipment.

Preferably, the equipment further includes a power supply input port formed in the lower shell for charging the power supply of the equipment or directly providing power supply for the equipment, foot pads 13 are arranged at the bottom of the equipment so as to beneficially improve the air inlet effect of the air inlets, meanwhile antiskid structures are arranged outside the foot pads to improve the product stability. The equipment can be provided with a power supply inside or is directly powered by an external power supply.

The control functions of the equipment mainly include gear control, indication control and spraying control, wherein the gear control includes the gear control at multiple time, the control of spraying size and other functions; the indication control refers to changing different colors by the light guide plate to achieve a lamplight indication effect; and the spraying control can achieve single spraying and double spraying.

The equipment has two sets of independent ultrasonic atomization devices, which can work simultaneously or separately at the same time, different types of essential oils are added to different water tanks, and different needs of users are satisfied by the simultaneous or separate work; if no essential oil is placed, the atomization on both sides is opened to the maximum gear at the same time to achieve the effect of a humidifier; a core atomization oscillator circuit uses a high-quality IC to generate variable frequency oscillation, in order to obtain better atomization experience, but also to extend the working life of the equipment; the machine is equipped with lamps with 7 colors, and different modes can be set to create different lamplight atmospheres; and manual, mobile APP and other control operations can be achieved, and voice control (Amazon ECHO and Google HOME) is supported.

The foregoing descriptions are only preferred embodiments of the present invention and are not intended to limit the present invention. Any modifications, equivalent replacements and improvements made within the spirit and principle of the present invention should be encompassed within the protection scope of the present invention.

1. Spraying equipment with a double-water-tank structure, comprising an upper shell and a lower shell, wherein two air outlets are formed in the top of the upper shell; two air inlets are formed in the bottom of the lower shell, a redundant space is arranged between the upper shell and the lower shell, the double-water-tank structure, a double-atomization-sheet module and double fans are arranged in the redundant space, meanwhile the two air outlets in the upper shell and the two air inlets in the lower shell form two sets of independent aromatherapy spraying equipment together; a control motherboard, a control device, a wireless device, a light guide plate and control keys are further arranged in the redundant space; the double-water-tank structure is arranged at an upper part of the control motherboard, the double-atomization-sheet module, the control device and the wireless device are installed on the control motherboard, and the double fans are installed at a lower part of the double-atomization-sheet module; and the control device is connected with the double-atomization-sheet module, the double fans, the wireless device, the light guide plate and the control keys, controls the equipment and achieves a variety of control functions.

2. The spraying equipment with the double-water-tank structure of claim 1, wherein the wireless device is connected with an intelligent control terminal, controls the equipment through system control software or other equipment software, and achieves a variety of control functions.

3. The spraying equipment with the double-water-tank structure of claim 1, wherein the control keys are composed of two or more keys, the control keys are touch keys, and different touch keys are touched to control the equipment and achieve a variety of control functions.

4. The spraying equipment with the double-water-tank structure of claim 1, wherein the double-atomization-sheet module generates ultrasonic waves after being energized, and the ultrasonic waves convert liquid substance in the water tanks into fog.

5. The spraying equipment with the double-water-tank structure of claim 1, wherein the fans are located below the water tanks, thereby being conducive to guiding the flow of the fog in the water tanks along the direction of the air outlets.

6. The spraying equipment with the double-water-tank structure of claim 1, wherein the light guide plate is connected with the control device and achieves different lamplight effects under different control functions so as to create environments with different atmospheres.

7. The spraying equipment with the double-water-tank structure of claim 1, further comprising a power supply input port formed in the lower shell for charging the power supply of the equipment or directly providing power supply for the equipment, wherein foot pads are arranged at the bottom of the equipment so as to beneficially improve the air inlet effect of the air inlets, meanwhile antiskid structures are arranged outside the foot pads to improve the product stability.
